(19) United States
(12) Patent Application Publication
(10) Pub. No.: US 2017/0202767 A1
(43) Pub. Date: Jul. 20, 2017

(54) Topical skin care formulations

(71) Applicant: Louise Holyfield, Dallas, TX (US)

(72) Inventor: Louise Holyfield, Dallas, TX (US)

(21) Appl. No.: 14/998,566

(22) Filed: Jan. 19, 2016

(51) Int. Cl. A61K 8/60; A61K 8/35; A61K 8/34; A61Q 19/04

(52) U.S. Cl. CPC A61K 8/60; A61Q 19/04; A61K 8/35; A61K 8/347

(57) ABSTRACT

A color-stable topical gel containing dihydroxyacetone, erythrulose, and vanillin in a sunless tanning, self-tanning, anti-aging, anti-oxidant formulation that gives the skin a longer lasting tan, without the use of dyes, stains or colorants.

### FIELD OF INVENTION

This invention relates to chemical and natural compositions, and more particularly to topical self-tanning, sunless tanning, anti-aging, anti-oxidant skin care formulations and methods of use.

### BACKGROUND OF THE INVENTION

Each year in the United States there are more new cases of skin cancer than the combined incidence of cancers of the breast, prostate, lung and colon. One person dies every 57 seconds from melanoma skin cancer. Statistics state the largest number of newly diagnosed cases of melanoma is in young adults ages 15 to 29.

Unfortunately, young adults are the most unlikely individuals to have their skin checked for cancer by a dermatologist compared with an older individual or someone who has previously been diagnosed with skin cancer.

Based on worldwide statistics Northern America leads the world in new cases of cancer each year.

About 86 percent of Melanomas can be attributed to exposure to ultraviolet (UV) radiation from the sun. About 90 percent of Basal and Squamous Cell Carcinomas can be attributed to exposure to ultraviolet (UV) radiation from the sun.

Melanoma accounts for less than two percent of skin cancer cases compared with Basal and Squamous Cell Carcinomas, but accounts for the vast majority of skin cancer deaths. If Melanoma goes undetected the patient will present with internal metastases of the cancer. Melanocytes also are present in other areas like the eyes for instance.

Statistics report the inherent danger of getting skin cancer as a result of overexposure to the sun, and through the use of tanning beds. Unfortunately it does not deter most individuals from using such methods to attain a tan. Damage to the skin from these methods of tanning is cumulative and irreversible.

Self-tanning, sunless tanning products currently in the marketplace use Dihydroxyacetone (DHA) along with dyes and stains. They are dark in color, extremely messy, difficult to apply, requires exfoliation of the skin prior to use, and will cause permanent staining to clothing upon contact.

Dihydroxyacetone, referred to as DHA, is the most common and widely known ingredient used in sunless tanning products. It is considered safe according to the Food, Drug & Cosmetic Industry (FD&C), It has been used as a color additive in the preparation of tanning products and cosmetics for almost 30 years.

Erythrulose is a common and widely known ingredient used in self-tanning and sunless tanning products. It is often used in combination with Dihydroxyacetone (DHA).

### SUMMARY OF THE INVENTION

A color stable topical gel comprising Dihydroxyacetone (DHA), Erythrulose, and Vanillin in a self-tanning, sunless tanning, anti-aging, anti-oxidant aqueous formulation that tans the skin for a longer period of time without the use of dyes, stains, or colorants.

One aspect of this invention provides a color stable topical gel that is a self-tanning, sunless tanning, anti-aging, anti-oxidant aqueous formulation comprising Dihydroxyacetone (DHA), Erythrulose, and Vanillin, or any known derivatives thereof.

A second aspect of the invention provides a color stable topical gel that is a self-tanning, sunless tanning, anti-aging, anti-oxidant aqueous formulation consisting of Dihydroxyacetone (DHA), Erythrulose, and Vanillin that does not contains dyes, stains or colorants.

A third aspect of the invention provides a color stable topical gel that is a self-tanning, sunless tanning, anti-aging, anti-oxidant aqueous formulation comprising Dihydroxyacetone (DHA), Erythrulose, and Vanillin that does not permanently stain clothing, linens, etc.

A fourth aspect of the invention provides a color stable topical gel that is a self-tanning, sunless tanning, anti-aging, anti-oxidant aqueous formulation consisting of Dihydroxyacetone (DHA), Erythrulose, and Vanillin that is superior in its ability to tan the first layer of skin, the stratum corneum and epidermis, for a longer period of time. The tan will not streak, and will fade evenly over time.

A fifth aspect of the invention provides a color stable topical gel that is a self-tanning, sunless tanning, anti-aging, anti-oxidant aqueous formulation comprising Dihydroxyacetone (DHA), Erythrulose, and Vanillin and does not require the skin be exfoliated prior to application of the product.

A sixth aspect of the invention provides a color stable topical gel that is a self-tanning, sunless tanning, anti-aging, anti-oxidant aqueous formulation containing Dihydroxyacetone (DHA), Erythrulose, and Vanillin for use in a spa setting where a professional massage therapist applies the tanning formulation while giving a massage at the same time.

A seventh aspect of the invention provides a color stable topical gel that is a self-tanning, sunless tanning, anti-aging, anti-oxidant aqueous formulation containing Dihydroxyacetone (DHA), Erythrulose, and Vanillin; and a method that includes the step of applying the product evenly all over the body and face with a sponge or polyeurethane foam pad.

An eighth aspect of the invention provides a color stable topical get that is a self-tanning, sunless tanning, anti-aging, anti-oxidant aqueous formulation containing from 0.05% to 15% Dihydroxyacetone (DHA), and from 0.05% to 2% Erythrulose, and from 0.05% to 15% Vanillin. All ingredient ratios within these percentages are useful. The use of larger percentages of Vanillin will result in a visibly darker gel. Many well-known aqueous cosmetic gels are useful in the formulations of the invention. See for example published Application No. PCT/US 1992/008743 and U.S. Pat. No. 4,863,725 A.

A ninth aspect of the invention provides a color stable topical get that is a self-tanning, sunless tanning, anti-aging, anti-oxidant aqueous formulation containing Dihydroxyacetone (DHA), Erythrulose, and Vanillin that has a very pleasant smell, unlike existing self-tanning, sunless tanning products that are well known for their unpleasant smell.

### EXAMPLES

A. 12% by wt. Dihydroxyacetone (DHA)
1% by wt. Erythrulose
4% by wt. Vanillin
83% by wt. aqueous gel
B. 15% by wt. Dihydroxyacetone (DHA)
2% by wt. Erythrulose
6% by wt. Vanillin
77% by wt. aqueous gel
C. 6% by wt. Dihydroxyacetone (DHA)
0.05% by wt. Erythrulose
3% by wt. Vanillin
90.5% by wt. aqueous gel
D. 9% by wt. Dihydroxyacetone (DHA)
1% by wt. Erythrulose
3% by wt. Vanillin
87% by wt. aqueous gel
E. 1% by wt. Dihydroxyacetone (DHA) p1 0.05% by wt. Erythrulose
1% by wt. Vanillin
97.5% by wt. aqueous gel
F. 3% by wt. Dihydroxyacetone (DHA)
0.05% by wt. Erythrulose
1% by wt. Vanillin
95.5% by wt. aqueous gel

The percentages of Dihydroxyacetone (DHA), Erythrulose, and Vanillin will vary depending on the color of tan desired, be it light, medium, dark, or very dark.

Preferred percentages for dark are 12% to 15 wt % Dihydroxyacetone (DHA), 1.0 to 2.0 wt % Erythrulose, and 4 to 6 wt % Vanillin. For medium the preferred percentages are 6 to 9 wt % Dihydroxyacetone (DHA), 0.5 to 1 wt % Erythrulose, and 3 to 4 wt % Vanillin. For light the preferred percentages are 1 to3 wt % Dihydroxyacetone (DHA), 05% Erythrulose, and 1 to 2 wt % Vanillin.

Preferred percentages for very dark are 12% Dihydroxyacetone (DHA), 2% Erythrulose, and 8% Vanillin. Preferred percentages for extremely dark are 15% Dihydroxyacetone (DHA), 2% Erythrulose, and 10% Vanillin.

Preferred percentages for light/maintenance is 1% Dihydroxyacetone (DHA), 0.05% Erythrulose, and 0.05% Vanillin.

1-4. (canceled)

5. A topical skin care formulation for self-tanning consisting essentially of an aqueous gel,
an effective amount of dihydroxyacetone for tanning the skin,
an effective amount of erythrulose for tanning the skin, and
an effective amount of vanillin for tanning the skin,
wherein the percentage of each ingredient is selected to provide the desired shade of tan, as follows:
for a light tan, 1 to 3 wt % dihydroxyacetone, 0.5 to 1.0 wt % erythrulose, and 1 to2 wt % vanillin;
for a medium tan, 6 to 9 wt % dihydroxyacetone, 0.5 to 1.0 wt % erythrulose, and 3 to 4 wt % vanillin;
for a dark tan, 12 to 15 wt% dihydroxyacetone, 1.0 to 2.0 wt % erythrulose, and 4 to 6 wt % vanillin.

6. A topical skin care formulation for self-tanning, consisting essentially of:
1 to 15 wt % dihydroxyacetone,
0.5 to 2 wt % erythrulose,
1 to 6 wt % vanillin, and
68 to 98.5 wt % aqueous gel.

7. A method for tanning the skin comprising the step of applying the formulation of claim 5 to the skin.

8. A method for tanning the skin comprising the step of applying the formulation of claim 6 to the skin.

9. A formulation as in claim 6 wherein the percentages are:
12 wt % dihydroxyacetone,
1 wt % erythrulose,
4 wt % vanillin, and
83 wt % aqueous gel.

10. A formulation as in claim 6 wherein the ingredient percentages are:
15% by wt. Dihydroxyacetone (DHA)
2% by wt. Erythrulose
6% by wt. Vanillin and
77% by wt. aqueous gel.

11. A formulation as in claim 6 wherein the ingredient percentages are:
6% by wt. Dihydroxyacetone (DHA)
0.05% by wt. Erythrulose
3% by wt. Vanillin and
90.5% by wt. aqueous gel.

12. A formulation as in claim 6 wherein the ingredient percentages are:
9% by wt. Dihydroxyacetone (DHA)
1% by wt. Erythrulose
3% by wt. Vanillin and
87% by wt. aqueous gel.

13. A formulation as in claim 6 wherein the ingredient percentages are :
1% by wt. Dihydroxyacetone (DHA)
0.05% by wt. Erythrulose
1% by wt. Vanillin

14. A formulation as in claim 6 wherein the ingredient percentages are:
3% by wt. dihydroxyacetone
0.05% by wt. erythrulose
1% by wt. vanillin and
95.5% by wt. aqueous gel.

15. A method as in claim 6 further including the step of having a massage at the same time.
